(19) United States
(12) Patent Application Publication
(10) Pub. No.: US 2026/0193591 A1
(43) Pub. Date: Jul. 9, 2026

(54) USE OF RAPE POLLEN IN PROMOTING HIGH-YIELD PRODUCTION OF MANACOLIN K BY MONASCUS PURPUREUS AND METHOD FOR PROMOTING HIGH-YIELD PRODUCTION OF MANACOLIN K

(71) Applicant: CHINESE ACADEMY OF AGRICULTURAL SCIENCES BEE INSTITUTE, Beijing (CN)

(72) Inventors: Wenli TIAN, Beijing (CN); Xiangxin LI, Beijing (CN); Jun ZHANG, Beijing (CN); Xiaoming FANG, Beijing (CN); Hualei CHEN, Beijing (CN); Fei PAN, Beijing (CN); Yaxi ZHOU, Beijing (CN); Xinning WU, Beijing (CN)

(21) Appl. No.: 19/341,519

(22) Filed: Sep. 26, 2025

Related U.S. Application Data
(63) Continuation of application No. PCT/CN2025/118985, filed on Sep. 4, 2025.

(30) Foreign Application Priority Data
Jan. 3, 2025 (CN) 202510008409.6

(51) Int. Cl. C12N 1/14

(52) U.S. Cl. CPC C12N 1/14

(57) ABSTRACT

The present invention provides use of rape pollen in promoting high-yield production of monacolin K by Monascus purpureus and a method for promoting high-yield production of monacolin K, and relates to the technical field of culture of Monascus purpureus. The method includes adding 0.6%-4.8% of rape pollen into a fermentation culture medium for fermentation culture of Monascus purpureus. The present invention overcomes deficiencies of the prior art. By adding the rape pollen into the culture medium of Monascus purpureus, the content of monacolin K can be significantly increased after culture for 15 days, and particularly, when an addition amount of the rape pollen is 2.4%, the content of monacolin K is increased by 3.50 times on day 15, reaching 1,524 mg/L.

### CROSS-REFERENCE TO RELATED APPLICATIONS

The present application is a continuation of International Application No. PCT/CN2025/118985, filed on Sep. 4, 2025, and claims priority to Chinese patent application No. 202510008409.6, titled “USE OF RAPE POLLEN IN PROMOTING HIGH-YIELD PRODUCTION OF MANACOLIN K BY MONASCUS PURPUREUS AND METHOD FOR PROMOTING HIGH-YIELD PRODUCTION OF MANACOLIN K”, filed on Jan. 3, 2025, the entire contents of which are incorporated herein by reference.

### FIELD OF TECHNOLOGY

The present invention relates to the technical field of culture of Monascus purpureus, and specifically relates to use of rape pollen in promoting high-yield production of monacolin K by Monascus purpureus and a method for promoting high-yield production of monacolin K.

### BACKGROUND

Monascus is a filamentous fungus capable of producing a variety of secondary metabolites with application values, such as Monascus pigments, monacolin K, γ-aminobutyric acid, etc. Among them, the monacolin K, also known as “lovastatin”, is a natural statin substance produced by Monascus, and has good lipid-lowering activity.

The ability of Monascus to synthesize the monacolin K through fermentation is the key to determining the quality of functional Monascus. However, existing Monascus has a relatively low capacity of monacolin K, resulting in a correspondingly increased industrial production cost, which severely restricts development of the functional Monascus industry and becomes a major obstructive factor for application of Monascus in fields such as food and health care products. In recent years, researchers have conducted extensive and in-depth studies on optimizing fermentation conditions, such as improving the composition and ratio of ingredients in culture media to promote the production of monacolin K by Monascus. For example, according to Zhang Chan, et al., synthesis of the monacolin K is increased by adding different concentrations of substances such as glutamic acid, arginine, honeysuckle, and nicotinamide into culture media. However, different exogenous additives are not always positively correlated with an increase in the monacolin K. Therefore, a large number of experiments need to be carried out to screen promoting substances.

### SUMMARY

In view of deficiencies of the prior art, the present invention provides use of rape pollen in promoting high-yield production of monacolin K by Monascus purpureus and a method for promoting high-yield production of monacolin K. The yield of monacolin K is promoted by adding rape pollen into a culture medium.

To achieve the above objective, the present invention is achieved through the following technical solution:
using rape pollen for promoting high-yield production of monacolin K by Monascus purpureus.

Preferably, the method includes adding rape pollen with a mass percentage of 0.6%-4.8% into a fermentation culture medium, and then inoculating Monascus purpureus into the fermentation culture medium for culture.

Preferably, the rape pollen with the mass percentage of 2.4% is added into the fermentation culture medium.

Preferably, the fermentation culture medium is an ordinary liquid fermentation culture medium, and calculated according to contents of ingredients per liter, a formula includes: 90 g of glycerol, 0.2 g of ZnSO₄·7H₂O, 20 g of rice flour, 2.5 g of KH₂PO₄, 10 g of peptone, 5 g of NaNO₃, 1 g of MgSO₄·7H₂O, and the balance of water.

The use of rape pollen in promoting high-yield production of monacolin K by Monascus purpureus and the method for promoting high-yield production of monacolin K provided by the present invention, compared with the prior art, have the following advantages:
by adding the rape pollen into the culture medium of Monascus purpureus, the content of monacolin K can be significantly increased after culture for 15 days, and particularly, when an addition amount of the rape pollen is 2.4%, the content of monacolin K is increased by 3.50 times on day 15, reaching 1,524 mg/L.

### BRIEF DESCRIPTION OF THE DRAWINGS

FIG. 1 is a bar comparison chart of yields of monacolin K produced at different times by Monascus purpureus M1 after fermentation culture in culture media with different contents of rape pollen in Example 1 of the present invention;

FIG. 2 is a bar comparison chart of yields of monacolin K produced at different times by Monascus purpureus M1 after fermentation culture in culture media with different contents of rape pollen in Example 2 of the present invention;

FIG. 3 is a bar comparison chart of yields of monacolin K of an experimental group and a control group in Example 3 of the present invention;

FIG. 4 is a comparison chart of color levels of yellow pigment of the experimental group and the control group in Example 3 of the present invention;

FIG. 5 is a comparison chart of color levels of orange pigment of the experimental group and the control group in Example 3 of the present invention;

FIG. 6 is a comparison chart of color levels of red pigment of the experimental group and the control group in Example 3 of the present invention;

FIG. 7 is a comparison chart of biomass of the experimental group and the control group in Example 3 of the present invention;

FIG. 8 is a comparison chart of pH changes of the experimental group, the control group, and a blank group in Example 3 of the present invention; and

FIG. 9 shows electron microscopy images of morphology of Monascus M1 cultured in the experimental group and the control group in Example 3 of the present invention, in which a and b show mycelial morphology in an ordinary culture medium; c and d show mycelial morphology in a rape pollen culture medium; a and c are magnified by 1000×; b and d are magnified by 10000×; and a red arrow indicates a wrinkle.

### DESCRIPTION OF THE EMBODIMENTS

To make the objectives, technical solutions, and advantages of the embodiments of the present invention clearer, the technical solutions in the embodiments of the present invention are described clearly and completely below in conjunction with the embodiments of the present invention. Obviously, the described embodiments are a part of the embodiments of the present invention, rather than all of the embodiments. Based on the embodiments of the present invention, all other embodiments obtained by those of ordinary skill in the art without creative efforts are within the scope of protection of the present invention.

### Example 1

Detection of monacolin K produced by culture of a wild-type Monascus purpureus strain M1 (commercially available, preservation No. CGMCC 3.0568)
1. An ordinary liquid fermentation culture medium includes ingredients as follows (g/L): 90 g of glycerol, 0.2 g of ZnSO₄·7H₂O, 20 g of rice flour, 2.5 g of KH₂PO₄, 10 g of peptone, 5 g of NaNO₃, and 1 g of MgSO₄·7H₂O.
2. Strain activation: The Monascus purpureus M1 was cultured on a PDA culture medium at 30° C. for 3 days and activated for 2 generations.
3. Preparation of a seed solution: 10 ml of sterile water was injected into the above PDA culture medium, mycelia on a surface of the culture medium were scraped with a sterile inoculation loop and then filtered through 2 layers of sterile filter paper, and a spore suspension was inoculated into an ordinary liquid seed culture medium at an inoculation amount of 5% and cultured at 30° C. and 200 r/min for 2 days.
4. Preparation of fermentation solutions: The seed solution was respectively inoculated into ordinary liquid fermentation culture media added with 0%, 1%, 2%, 5%, 10%, and 20% of rape pollen at an inoculation amount of 10%, cultured at 30° C. and 150 r/min for 2 days, and then continuously cultured at 25° C. and 150 r/min.

Contents of monacolin K in the culture media of various groups were detected.

A specific detection process is as follows.

Pretreatment of the fermentation solutions: 2 mL of the fermentation solution in each group was weighed, added into 6 mL of 75% methanol, subjected to ultrasonic extraction for 30 minutes and standing overnight, then filtered through a 0.22 μm filter membrane into a vial for liquid chromatography, and stored in a refrigerator at 4° C.

Detection of monacolin K: A high performance liquid chromatography (HPLC) method was adopted using a chromatographic column of Inertsi10DS-3 C18 (150 mm×4.6 mm×5 μm), a mobile phase including 0.1% phosphoric acid and methanol at 1:3 at a flow rate of 1 mL/min, and a detector being ultraviolet detector (PDA) at a detection wavelength of 237 nm, a detection temperature of 30° C., and an injection volume of 10 μL.

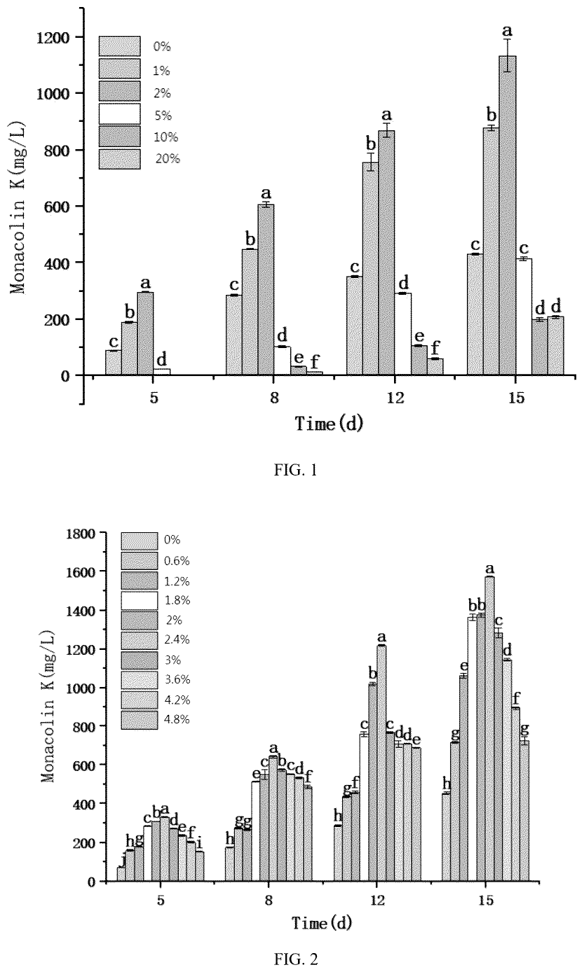

Detection results are shown in FIG. 1. When the addition amount of the rape pollen is 5%, the content of monacolin K after culture for 15 days is decreased compared with that without addition of the rape pollen.

### Example 2

Referring to Example 1, the seed solution was respectively inoculated into ordinary liquid fermentation culture media added with 0%, 0.6%, 1.2%, 1.8%, 2%, 2.4%, 3%, 3.6%, 4.2%, and 4.8% of rape pollen at an inoculation amount of 10%, cultured at 30° C. and 150 r/min for 2 days, and then continuously cultured at 25° C. and 150 r/min.

Contents of monacolin K in the culture media of various groups were detected. Specific results are shown in FIG. 2. In the ordinary liquid fermentation culture medium added with 2.4% of the rape pollen, the content of monacolin K is the highest on day 15, and the content of monacolin K is increased by 3.50 times compared with that of the culture medium added with 0% of the rape pollen, reaching 1,524 mg/L.

### Example 3

Referring to Example 2, the seed solution was respectively inoculated into ordinary liquid fermentation culture media added with 0% and 2.4% of rape pollen at an inoculation amount of 10%, the culture medium added with 0% of the rape pollen for culture was set as a control group, the culture medium added with 2.4% of the rape pollen for culture was set as an experimental group, and a pure ordinary liquid fermentation culture medium without inoculation treatment was set as a blank group. The seed solution was cultured at 30° C. and 150 r/min for 2 days, and then continuously cultured at 25° C. and 150 r/min. The following detections were carried out.

### 1. Detection of Monacolin K:

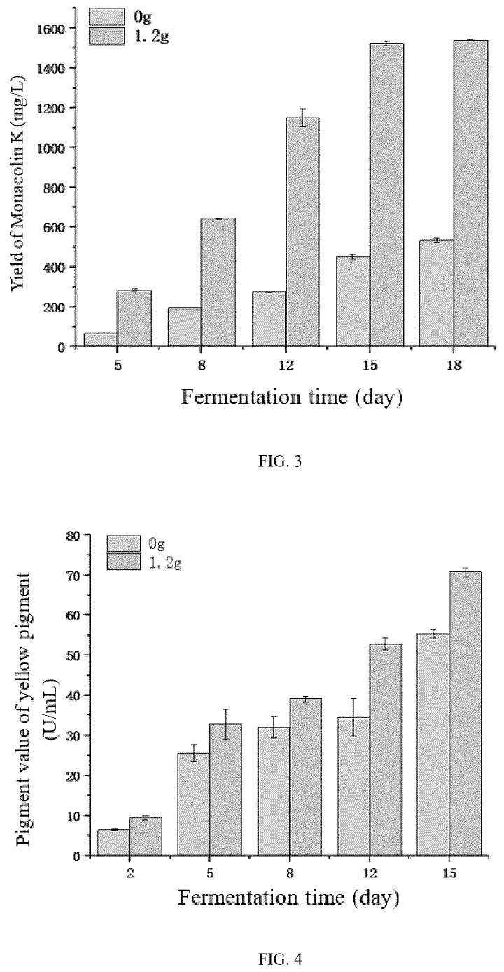

Referring to the detection method in Example 1, contents of monacolin K of the control group and the experimental group at different culture times were detected. Specific results are shown in FIG. 3. After fermentation culture for 5-18 days, the content of monacolin K in the experimental group is significantly higher than that in the control group.

### 2. Detection of Pigment Value

Pretreatment of fermentation solutions: 1 ml of the fermentation solution in each group was weighed, added into 6 ml of a 70% ethanol solution, then placed in a thermostatic water bath pot for water bath treatment at 60° C. for 1 hour, centrifuged at 4,000 rpm for 15 minutes, and stored in the dark.

Detection of pigment value: Absorbance values at 410, 448, and 505 nm were measured using an ultraviolet spectrophotometer.

A quantification formula is as follows: pigment value of Monascus pigment (U/mL)=absorbance value×dilution factor.

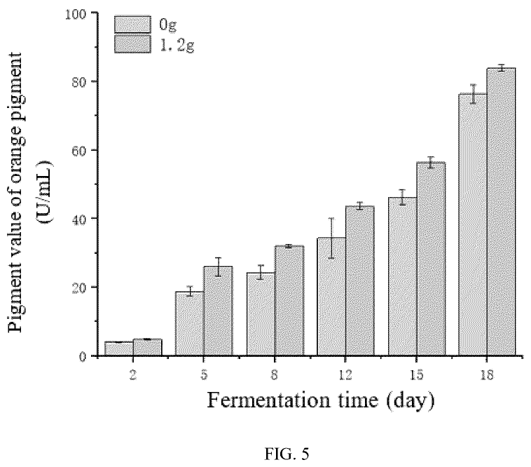

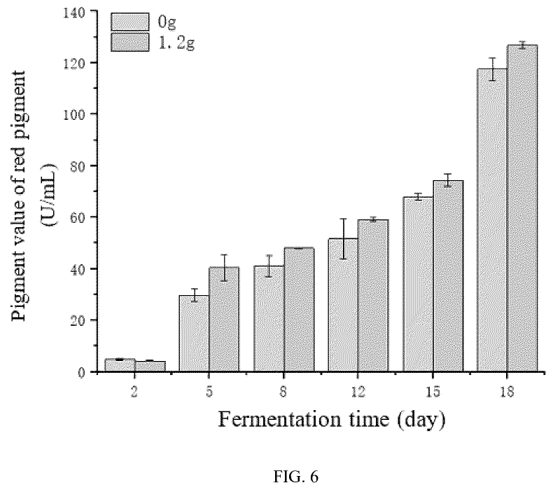

Results are shown in FIG. 4, FIG. 5, and FIG. 6, where the pigment value of yellow pigment, the pigment value of orange pigment, and the pigment value of red pigment in the experimental group are significantly higher than those in the control group after fermentation culture for 5 days.

### 3. Detection of Biomass

The biomass of mycelia was measured using a dry weight method. 3 mL of the fermentation solution in each group was weighed, added into a centrifuge tube, then transferred to filter paper for filtration, and washed 3 times with ultrapure water. After drying as much as possible, the mycelia were placed in an oven for drying to a constant weight at 60° C. to obtain the constant weight of the mycelia.

$$Biomass ( g / L ) = ( dry matter weight / fermentation solution volume ) \times 1000.$$

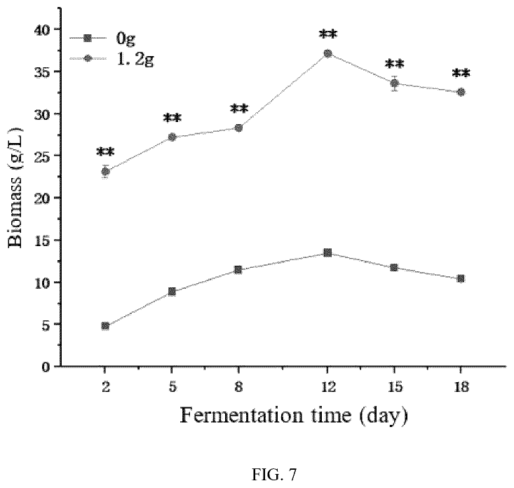

Results are shown in FIG. 7, where the biomass of the experimental group is significantly higher than that of the control group.

### 4. Detection of pH Value

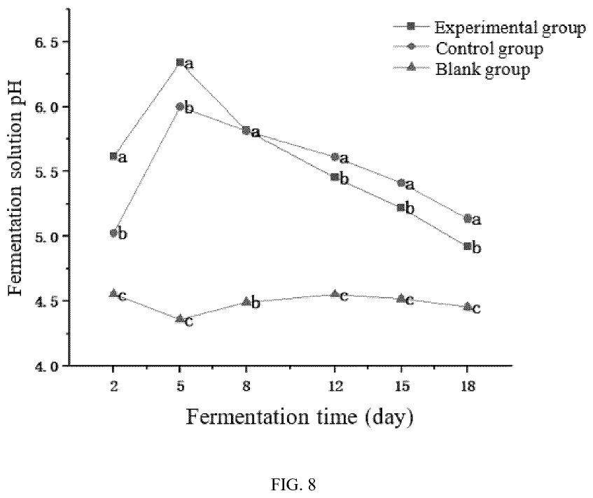

Detection was performed using a pH meter. Specific results are shown in FIG. 8. When the fermentation time is less than 8 days, the pH of the experimental group is higher than that of the control group. When the fermentation time is more than 8 days, the pH of the experimental group is lower than that of the control group.

### 5. Scanning Electron Microscopy Processing

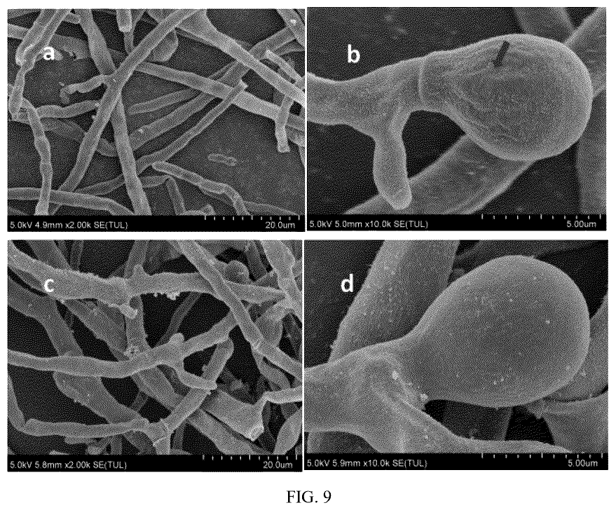

The Monascus mycelia cultured for 12 days in each group were centrifuged at 12,000 r/min for 5 minutes to collect mycelium cells, and the cells were resuspended (blown with a pipette tip to avoid cell loss caused by the cells sucked into the pipette tip during blowing) in a 2.5% glutaraldehyde solution (diluted with a PBS buffer solution) for fixation for 12 hours. The cells were rinsed twice (resuspended and centrifuged twice) with a 0.1 M phosphate buffer solution (PBS, pH 7.2), and a supernatant was discarded. The cells were dehydrated sequentially with ethanol solutions of different concentrations (30%, 50%, 70%, 80%, 90%, 100%), followed by standing at each concentration for 10 minutes and centrifugation at 12,000 r/min for 5 minutes (each concentration was repeated twice), and a supernatant was discarded. First, the mycelia were resuspended in isopentyl acetate and ethanol (v:v=1:1), and then resuspended in isopentyl acetate to replace the ethanol in the cells. The cells were resuspended in each solvent for 10 minutes and centrifuged at 12,000 r/min for 5 minutes, and a supernatant was discarded. Hexamethyl disilazane (HMDS) as a solvent was added to cover samples, tops of centrifuge tubes were sealed with absorbent cotton to improve water absorbency, and the samples were placed in an oven for drying at 60° C. until the samples became powdery. The samples were sent for observation by scanning electron microscopy. Specific results are shown in FIG. 9, in which a and b show mycelial morphology in the control group; c and d show mycelial morphology in the experimental group; a and c are magnified by 1000×; b and d are magnified by 10000×; and a red arrow indicates a wrinkle, indicating that the Monascus mycelia cultured in the experimental group have better morphology.

The above embodiments are only used to illustrate the technical solutions of the present invention and are not to be construed as limitations. Although the present invention has been described in detail with reference to the above embodiments, those of ordinary skill in the art should understand that they can still modify the technical solutions described in the above embodiments or make equivalent substitutions for some technical features therein. These modifications or substitutions are made without making the essence of the corresponding technical solutions departed from the spirit and scope of the technical solutions of the embodiments of the present invention.

What is claimed is:

1. Use of rape pollen in promoting high-yield production of monacolin K by Monascus purpureus.

2. A method for promoting high-yield production of monacolin K by Monascus purpureus, comprising: adding rape pollen with a mass percentage of 0.6%-4.8% into a fermentation culture medium, and then inoculating Monascus purpureus into the fermentation culture medium for culture.

3. The method for promoting high-yield production of monacolin K by Monascus purpureus according to claim 2, wherein the rape pollen with the mass percentage of 2.4% is added into the fermentation culture medium.

4. The method for promoting high-yield production of monacolin K by Monascus purpureus according to claim 2, wherein the fermentation culture medium is an ordinary liquid fermentation culture medium, and calculated according to contents of ingredients per liter, a formula comprises: 90 g of glycerol, 0.2 g of ZnSO₄·7H₂O, 20 g of rice flour, 2.5 g of KH₂PO₄, 10 g of peptone, 5 g of NaNO₃, 1 g of MgSO₄·7H₂O, and the balance of water.
